(19) United States
(12) Patent Application Publication
(10) Pub. No.: US 2005/0107257 A1
(43) Pub. Date: May 19, 2005

(54) Derivatives of (1-benzyl-piperidine-4-yl)-diphenyl-methanol and their use as pesticide

(76) Inventors: Saleem Farooq, Arisdorf (CH); Stephen Trah, Basel (CH); Andre Jeanguenat, Basel (CH)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/502,634

(22) PCT Filed: Feb. 10, 2003
(86) PCT No.: PCT/EP03/01299

(30) Foreign Application Priority Data
Feb. 11, 2002 (CH) 217/02

(51) Int. Cl. A01N 43/40; C07D 211/26; C07D 211/60

(52) U.S. Cl. 504/248; 546/229; 546/230; 546/239

(57) ABSTRACT

Compounds of formula (I), wherein R₁ and R₂ are, for example, hydrogen, halogen, C₁-C₆-alkyl, C₃-C₆cycloalkyl, halo-C₁-C₆alkyl, C₁-C₆alkoxy or halo-C₁-C₆alkoxy; R₃ and R₄ are hydrogen or together form a bond; R₅ is, for example, C₁-C₆alkyl C₁-C₆alkyl, C₃-C₆cycloalkyl or C₂-C₄alkenyl; R₅₅ is, for example, hydrogen, C₁-C₆alkyl or halo-C₁-C₆alkyl; R₆ is, for example, hydrogen, halogen, CN, NO₂ C₁-C₆alkyl, halo-C₁-C₆alkyl, C₃-C₆cycloalkyl, halo-C₃-C₆cycloalkyl, C₃-C₆cycloalkoxy, C₁-C₆alkoxy, halo-C₁-C₆alkoxy, C₂-C₄alkenyl, C₂-C₄alkynyl or halo-C₂-C₄alkenyl; m is 1, 2, 3, 4 or 5; n is 1, 2, 3, 4, or 5; o is 1, 2, or 3: q is 0 or 1; s is 1, 2, 3, 4 or 5; and, where applicable, E/Z isomers, mixtures of E/Z isomers and/or tautomers, in each case in free form or in salt form; a process for the preparation of and the use of those compounds, pesticidal compositions in which the active ingredient has been selected from those compounds or an agrochemically acceptable salt thereof, a process for the preparation of and the use of those compositions, plant propagation material that has been treated with those compositions, and a method of controlling pests are described.

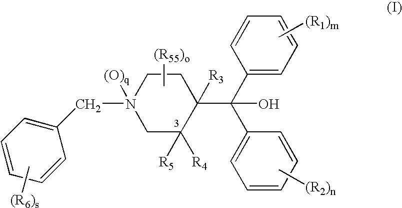

The present invention relates to (1) a compound of formula 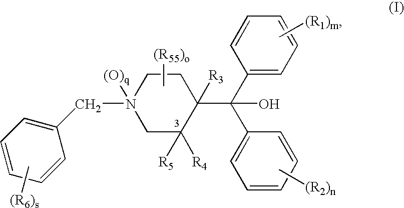
wherein
R₁ and R₂ are each independently of the other hydrogen, halogen, C₁-C₆alkyl, C₃-C₆-cycloalkyl, halo-C₁-C₆alkyl, halo-C₃-C₆cycloalkyl, C₂-C₄alkenyl, C₂-C₄alkynyl, halo-C₂-C₄alkenyl, halo-C₂-C₄alkynyl, C₁-C₆alkoxy, halo-C₁-C₆alkoxy, C₂-C₆alkenyloxy, C₂-C₆alkynyloxy, halo-C₂-C₆alkenyloxy, halo-C₂-C₆alkynyloxy, —SF₅, —C(═O)N(R₇)₂, —O—C(═O)N(R₇)₂, —CN, —NO₂, —S(═O)₂N(R₇)₂, —S(═O)ₚ—C₁-C₆alkyl, —S(═O)ₚ-halo-C₁-C₆alkyl, —O—S(═O)ₚ—C₁-C₆alkyl, —O—S(═O)ₚ-halo-C₁-C₆alkyl, phenyl, benzyl, phenoxy or benzyloxy, wherein each of the phenyl, benzyl, phenoxy or benzyloxy radicals is unsubstituted or is substituted in the aromatic ring by from one to five substituents selected independently of one another from the group consisting of halogen, cyano, NO₂, C₁-C₆alkyl, halo-C₁-C₆alkyl, C₁-C₆alkoxy and halo-C₁-C₆alkoxy;
R₃ and R₄ are hydrogen or together form a bond;
R₅ is C₁-C₆alkyl, halo-C₁-C₆alkyl, C₃-C₆cycloalkyl, C₂-C₄alkenyl, C₂-C₄alkynyl, C₁-C₆-alkoxy, C₁-C₆alkoxyalkyl, halo-C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, halogen or hydroxy;
R₅₅ is hydrogen, C₁-C₆alkyl, halo-C₁-C₆alkyl, C₃-C₆cycloalkyl, C₂-C₄alkenyl, C₂-C₄-alkynyl, C₁-C₆alkoxy, C₁-C₆alkoxyalkyl, halo-C₁-C₆alkoxy, C₂-C₆alkenyloxy or C₂-C₆alkynyloxy;
R₆ is hydrogen, halogen, CN, NO₂, C₁-C₆alkyl, halo-C₁-C₆alkyl, C₃-C₆cycloalkyl, halo-C₃-C₆cycloalkyl, C₃-C₆cycloalkoxy, C₁-C₆alkoxy, halo-C₁-C₆alkoxy, C₂-C₄alkenyl, C₂-C₄-alkynyl, halo-C₂-C₄alkenyl, halo-C₂-C₄alkynyl, C₂-C₆alkenyloxy, C₂-C₆alkynyloxy, halo-C₂-C₆-alkenyloxy, halo-C₂-C₆alkynyloxy, —C(═O)-C₁-C₆alkyl, —C(═O)-halo-C₁-C₆alkyl, —C(═O)—OC₁-C₆alkyl, —C(═O)—O-halo-C₁-C₆alkyl, —N(R₇)₂, —C(═O)N(R₇)₂, —O—C(═O)N(R₇)₂, —S(═O)₂N(R₇)₂, —S(═O)ₚ—C₁-C₆alkyl, —S(═O)ₚ-halo-C₁-C₆alkyl, —O—S(═O)ₚ—C₁-C₆alkyl, —O—S(═O)ₚ-halo-C₁-C₆alkyl, —NR₁₂—C(═Y)-Z-R₁₃, —C(R₉)═N—W—R₁₀,
benzyl, phenoxy, benzyloxy; or phenyl, benzyl, phenoxy, benzyloxy, heterocyclyl or heterocyclyloxy each of which is substituted by from one to five substituents selected independently of one another from the group consisting of halogen, cyano, NO₂, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈cycloalkoxy, C₃-C₈cycloalkoxy-C₁-C₆alkyl, C₃-C₈cycloalkyl-C₁-C₆alkoxy, halo-C₁-C₆alkoxy, C₂-C₄alkenyl, C₂-C₄alkynyl, halo-C₂-C₄alkenyl, halo-C₂-C₄alkynyl, C₂-C₆alkenyloxy, C₂-C₆alkynyloxy, halo-C₂-C₆alkenyloxy, halo-C₂-C₆alkynyloxy, —N(R₈)₂, phenyl, benzyl, phenoxy, benzyloxy, heterocyclyl and heterocyclyloxy;
the two R₇ radicals are each independently of the other hydrogen, C₁-C₁₋₂alkyl, halo-C₁-C₁₂alkyl, C₂-C₁₂alkenyl, halo-C₂-C₁₂alkenyl, C₂-C₁₂alkynyl, halo-C₂-C₁₂alkynyl, —C(═O)—R₁₀, —C(═S)—R₁₀, —C(═O)—O—R₁₀, —C(═S)—O—R₁₀, —C(═O)—NR₁₀R₁₁, —C(═S)—NR₁₀R₁₁, —S(═O)ₚ—R₁₀, C₃-C₈cycloalkyl, aryl, aryl-C₁-C₆alkyl, heterocyclyl, heterocyclyl-C₁-C₆alkyl; or C₃-C₉cycloalkyl, aryl, aryl-C₁-C₆alkyl, heterocyclyl or heterocyclyl-C₁-C₆alkyl which, depending upon the possibilities of substitution, are each substituted in the ring by from one to five substituents selected independently of one another from halogen, hydroxy, cyano, nitro, C₁-C₆alkyl, halo-C₁-C₆alkyl, C₁-C₆alkoxy and halo-C₁-C₆alkoxy; or
together, with the nitrogen atom to which they are bonded, form a heterocyclic ring that is unsubstituted or substituted;
R₈ is hydrogen, C₀-C₆alkyl or benzyl;
R₉ is halogen, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈cycloalkoxy, C₃-C₈cycloalkoxy-C₁-C₆alkyl, halo-C₁-C₆alkoxy, —NH(C₁-C₆-alkyl) or —N(C₁-C₆alkyl)₂;
R₁₀ and R₁₁ are each independently of the other hydrogen, C₁-C₆alkyl, C₃-C₈cycloalkyl, C₃-C₈cycloalkyl-C₁-C₆alkyl, halo-C₁-C₆alkyl, C₂-C₄alkenyl, C₂-C₄alkynyl, halo-C₂-C₄alkenyl, halo-C₂-C₄alkynyl or —C(═O)-C₁-C₆alkyl;
R₁₂ is hydrogen, C₁-C₆alkyl, C₁-C₆alkoxy-C₁-C₆alkyl, C₃-C₈cycloalkyl, halo-C₁-C₆alkyl, C₂-C₆alkenyl or C₂-C₆alkynyl;
R₁₃ is hydrogen, C₁-C₆alkyl, halo-C₁-C₆alkyl, C₁-C₆alkoxy-C₁-C₆alkyl, C₃-C₈cycloalkyl, halo-C₁-C₆alkyl, halo-C₃-C₈cycloalkyl, C₂-C₆alkenyl, C₂-C₆alkynyl, halo-C₂-C₆alkenyl, halo-C₂-C₆alkynyl, aryl, aryl-C₁-C₆alkyl or heterocyclyl, or aryl, aryl-C₁-C₆alkyl or heterocyclyl each of which is substituted by from one to three substituents selected from the group consisting of halogen, cyano, NO₂, C₁-C₆alkyl, C₃-C₈cycloalkyl, halo-C₁-C₆alkyl, C₁-C₆alkoxy, halo-C₁-C₆alkoxy, C₂-C₄alkenyl, C₂-C₄alkynyl, halo-C₂-C₄alkenyl, halo-C₂-C₄alkynyl, C₂-C₆alkenyloxy and C₂-C₆alkynyloxy;
m is 1, 2, 3, 4 or 5;
n is 1, 2, 3, 4 or 5;
o is 1, 2 or 3;
p is 0, 1 or 2;
q is 0 or 1;
s is 1, 2, 3, 4 or 5;
Y is O or S;
Z is a bond, O, S or NR₁₄;
R₁₄ is hydrogen, C₁-C₆alkyl, C₁-C₆alkoxy-C₁-C₆alkyl, C₃-C₈cycloalkyl, halo-C₁-C₆alkyl, C₂-C₆alkenyl or C₂-C₆alkynyl;
W is O or NH or N-C₁-C₆alkyl;
and, where applicable, to E/Z isomers, mixtures of E/Z isomers and/or tautomers, in each case in free form or in salt form;
to a process for the preparation of and to the use of those compounds, to pesticidal compositions in which the active ingredient has been selected from those compounds or an agrochemically acceptable salt thereof, to a process for the preparation of and to the use of those compositions, to plant propagation material that has been treated with those compositions, and to a method of controlling pests.

Certain piperidine derivatives are proposed in the literature as active ingredients in pesticides. The biological properties of those known compounds are not, however, entirely satisfactory in the area of pest control, for which reason there is a need to provide further compounds having pesticidal properties, especially for the control of insects and members of the order Acarina, that problem being solved according to the invention by the provision of the present compounds of formula (I).

The compounds of formula (I) and, where applicable, their tautomers can form salts, for example acid addition salts. These acid addition salts are formed, for example, with strong inorganic acids, such as mineral acids, for example sulfuric acid, a phosphoric acid or a hydrohalic acid, with strong organic carboxylic acids, such as unsubstituted or substituted, for example halo-substituted, C₁-C₄alkanecarboxylic acids, for example acetic acid, unsaturated or saturated dicarboxylic acids, for example oxalic acid, malonic acid, maleic acid, fumaric acid or phthalic acid, hydroxycarboxylic acids, for example ascorbic acid, lactic acid, malic acid, tartaric acid or citric acid, or benzoic acid, or with organic sulfonic acids, such as unsubstituted or substituted, for example halo-substituted, C₁-C₄alkane- or aryl-sulfonic acids, for example methane- or p-toluene-sulfonic acid. Compounds of formula (I) that have at least one acidic group can furthermore form salts with bases. Suitable salts with bases are, for example, metal salts, such as alkali metal salts or alkaline earth metal salts, for example sodium, potassium or magnesium salts, or salts with ammonia or with an organic amine, such as morpholine, piperidine, pyrrolidine, a mono-, di- or tri-lower alkylamine, for example ethylamine, diethylamine, triethylamine or dimethylpropylamine, or a mono-, di- or tri-hydroxy-lower alkylamine, for example mono-, di- or tri-ethanolamine. Corresponding internal salts may also be formed where appropriate. The free form is preferred. Among the salts of the compounds of formula (I), the agrochemically advantageous salts are preferred. Hereinbefore and hereinafter, any reference to the free compounds of formula (I) or their salts is to be understood as including, where appropriate, also the corresponding salts or the free compounds of formula (I), respectively. The same applies to tautomers of compounds of formula (I) and salts thereof.

Unless defined otherwise, the general terms used hereinbefore and hereinafter have the meanings given below.

Halogen—as a group per se and as a structural element of other groups and compounds, such as haloalkyl, halocycloalkyl, haloalkenyl, haloalkynyl and haloalkoxy—is fluorine, chlorine, bromine or iodine, especially fluorine, chlorine or bromine, more especially fluorine or chlorine, especially chlorine.

Unless defined otherwise, carbon-containing groups and compounds each contain from 1 up to and including 20, preferably from 1 up to and including 18, more especially from 1 up to and including 10, especially from 1 up to and including 6, more especially from 1 up to and including 4, especially from 1 up to and including 3, especially 1 or 2, carbon atoms, methyl being most especially preferred.

Alkyl—as a group per se and as a structural element of other groups and compounds, such as, for example, haloalkyl, alkoxy, alkoxyalkyl, haloalkoxy, alkoxycarbonyl, alkylthio, haloalkylthio, alkylsulfonyl and alkylsulfonyloxy—is, in each case giving due consideration to the number of carbon atoms contained in the group or compound in question, either straight-chained, e.g. methyl, ethyl, n-propyl, n-butyl, n-hexyl, n-octyl, n-decyl, n-dodecyl, n-hexadecyl or n-octadecyl, or branched, e.g. isopropyl, isobutyl, sec-butyl, tert-butyl, isopentyl, neopentyl or isohexyl.

Alkenyl and alkynyl—as groups per se and as structural elements of other groups and compounds, such as haloalkenyl, haloalkynyl, alkenyloxy, haloalkenyloxy, alkynyloxy or haloalkynyloxy—are straight-chained or branched and each contains two or preferably one unsaturated carbon-carbon bond(s). Examples are vinyl, prop-2-en-1-yl, 2-methylprop-2-en-1-yl, but-2-en-1-yl, but-3-en-1-yl, prop-2-yn-1-yl, but-2-yn-1-yl and but-3-yn-1-yl.

Cycloalkyl—as a group per se and as a structural element of other groups and compounds, such as, for example, alkyl—is cyclopropyl, cyclobutyl, cyclopentyl, cyclohexyl, cycloheptyl or cyclooctyl. Cyclopentyl and cyclohexyl, and especially cyclopropyl, are preferred.

Halo-substituted carbon-containing groups and compounds, such as haloalkyl and haloalkoxy, may be partially halogenated or perhalogenated, it being possible in the case of polyhalogenation for the halogen substituents to be the same or different. Examples of haloalkyl—as a group per se and as a structural element of other groups and compounds, such as haloalkoxy—are methyl substituted from one to three times by fluorine, chlorine and/or bromine, such as CHF₂, CF₃ or CH₂Cl; ethyl substituted from one to five times by fluorine, chlorine and/or bromine, such as CH₂CF₃, CF₂CF₃, CF₂CCl₃, CF₂CHCl₂, CF₂CHF₂, CF₂CFCl₂, CH₂CH₂C₁, CF₂CHBr₂, CF₂CHClF, CF₂CHBrF or CClFCHClF; propyl or isopropyl substituted from one to seven times by fluorine, chlorine and/or bromine, such as CH₂CHBrCH₂Br, CF₂CHFCF₃, CH₂CF₂CF₃, CF₂CF₂CF₃, CH(CF₃)₂ or CH₂CH₂CH₂Cl; and butyl or an isomer thereof substituted from one to nine times by fluorine, chorine and/or bromine, such as CF(CF₃)CHFCF₃, CF₂(CF₂)₂CF₃ or CH₂(CF₂)₂CF₃.

Aryl is especially phenyl or naphthyl, preferably phenyl.

Heterocyclyl is a 5- to 7-membered saturated or unsaturated ring which is preferably aromatic and which has from one to four hetero atoms selected from the group consisting of N, O and S. Preference is given to aromatic 5- and 6-membered rings that have a nitrogen atom as hetero atom and that may have a further hetero atom, preferably nitrogen or sulfur, especially nitrogen. Preferred heterocyclyl radicals are, for example, pyrrolyl, pyrazolyl, imidazolyl, 1,2,4-triazolyl, 1,2,4-oxadiazolyl, tetrazolyl, pyrazinyl, pyridyl, pyrimidinyl, pyridazinyl, thiazolyl, isothiazolyl, isoxazolyl, indolyl, indazolyl, benzimidazolyl, benzothiazolyl, furanyl, tetrahydrofuranyl and thienyl; preference is given to tetrazolyl, especially tetrazolyl that is substituted by C₁-C₃alkyl, especially methyl, ethyl, propyl or isopropyl, more especially by ethyl.

Preferred embodiments within the scope of the invention are
a compound according to the above group (1) of formula (I) wherein
R₁ and R₂ are each independently of the other halogen, C₁-C₂alkyl, C₃-C₆cycloalkyl, halo-C₁-C₂alkyl, C₁-C₂alkoxy, halo-C₁-C₂alkoxy, —C(═O)N(CH₃)₂, —CN or —NO₂;
especially are each independently of the other halogen, C₁-C₂alkyl, halo-C₁-C₂alkyl, C₁-C₂alkoxy or halo-C₁-C₂alkoxy;
more especially are each independently of the other chlorine, bromine, methyl, tri-fluoromethyl, methoxy or trifluoromethoxy;
even more especially are each independently of the other chlorine, trifluoromethyl or trifluoromethoxy;
most especially wherein the two substituents are CF₃, are in para-position and m and n are 1;
(3) a compound according to (1) or (2) of formula (I) wherein R₃ and R₄ are hydrogen;
especially wherein R₃ and R₅ are cis to each other;
(4) a compound according to (1) or (2) of formula (I) wherein R₃ and R₄ together form a bond;
a compound according to any one of groups (1) to (4) of formula (I) wherein
R₅ is C₁-C₆alkyl or halo-C₁-C₆alkyl; especially methyl or ethyl; more especially methyl;
a compound according to any one of groups (1) to (3) and (5) of formula (I) wherein o is 1 and R₅ is in the 3-position on the piperidine ring; especially wherein R₃ and R4 are hydrogen and R₃ and R₅ are in the cis-configuration;
(7) a compound according to any one of groups (1) to (6) of formula (I) wherein
R₆ is —NR₁₂—C(═Y)—Z—R₁₃ and R₁₃ is C₁-C₆alkyl, C₁-C₆alkoxy-C₁-C₆alkyl, C₃-C₈cycloalkyl, halo-C₁-C₆alkyl, halo-C₃-C₈cycloalkyl, C₂-C₆alkenyl, C₂-C₆alkynyl, halo-C₂-C₆alkenyl or halo-C₂-C₆alkynyl;
(8) a compound according to any one of groups (1) to (6) of formula (I) wherein
R₆ is —N(R₇)₂ and the two R₇ radicals are each independently of the other hydrogen, C₁-C₁₂alkyl, halo-C₁-C₁₂alkyl, C₂-C₁₂alkenyl, —C(═O)—R₁₀, —C(═S)—R₁₀, —C(═O)—O—R₁₀, —C(═S)—O—R₁₀, —C(═O)—NR₁₀R₁₁, —C(═S)—NR₁₀R₁₁ or —S(═O)ₚ—R₁₀,
especially wherein R₆ is —NHR₇ and R₇ is —C(═O)—O—R₁₀;
(9) a compound according to (8) of formula (I) wherein
R₁₀ and R₁₁ are each independently of the other hydrogen, C₁-C₆alkyl, C₃-C₈cycloalkyl, C₃-C₈cycloalkyl-C₁-C₆alkyl, halo-C₁-C₆alkyl, C₂-C₄alkenyl, C₂-C₄alkynyl, halo-C₂-C₄alkenyl, halo-C₂-C₄alkynyl or —C(═O)-C₁-C₆alkyl;
especially hydrogen, C₁-C₂alkyl, C₃-C₈cycloalkyl, C₁-C₂halogenalkyl or —C(═O)-C₁-C₆-alkyl;
(12) a compound according to (8) of formula (I) wherein
p is 1 or 2; especially 2;
(13) a compound according to (1) to (12) of formula (I) wherein
R₅₆ is hydrogen;
(14) a compound according to (1) to (13) of formula (I) wherein
q is 1;
(15) a compound according to (8) of formula (I) wherein
R₉ is halogen, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈cycloalkoxy, C₃-C₈cycloalkoxy-C₁-C₆alkyl, halo-C₁-C₆alkoxy, —NH(C₁-C₆-alkyl) or —N(C₁-C₆alkyl)₂;
(16) a compound according to (7) of formula (I) wherein
W is O or NH; especially O;
(17) a compound according to (1) to (6) and of formula (I) wherein
R₆ is heterocyclyl that is unsubstituted or substituted by from one to three substituents selected independently of one another from the group consisting of halogen, cyano, NO₂, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈-cycloalkoxy, C₃-C₈cycloalkoxy-C₁-C₆alkyl, C₃-C₈cycloalkyl-C₁-C₆alkoxy, halo-C₁-C₆alkoxy, C₂-C₄alkenyl, C₂-C₄alkynyl, halo-C₂-C₄alkenyl, halo-C₂-C₄alkynyl, C₂-C₆alkenyloxy, C₂-C₆-alkynyloxy, halo-C₂-C₆alkenyloxy, halo-C₂-C₆alkynyloxy and —N(R₈)₂, especially wherein R₆ is tetrazolyl that is unsubstituted or mono-substituted by C₁-C₆-alkyl.

Special preference is given within the scope of the invention to the compounds of formula (I) listed in the Tables.

The invention further relates to a process for the preparation of a compound of formula (I) wherein (R₁)ₘ, (R₂)ₙ, R₅, R₅, R₆, O, q and s are as defined for formula (I) and R₃ and R₄ together form a bond, which comprises
(a) reacting a compound of formula 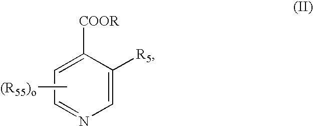
which is known or can be prepared by methods known per se and wherein R is C₁-C₈alkyl and R₅, R₅₅ and o are as defined for formula (I), with a compound of formula 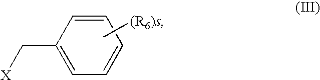
which is known per se and wherein (R₆)ₛ is as defined for formula (I) and X is a leaving group, to form a compound of formula 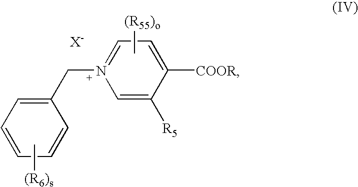
wherein R, R₅, R₅₅, R₆, s and o are as defined for formulae (II) and (III) and X is the anion of the said leaving group;
(b) reducing the resulting compound of formula (IV) to form a compound of formula 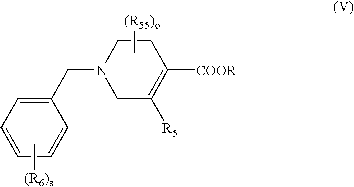
wherein R₁, R₅, R₅₅, R₆, o, s and R are as defined for formula (IV);
(c) either reacting the resulting compound of formula (V) with two moles of a compound of formula 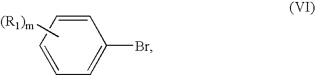
wherein (R₁)ₙ is as defined for formula (I), or
reacting the compound of formula (V) with one mole of a compound of formula (VI) and then with one mole of a compound of formula 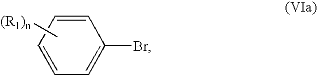
wherein (R₂)ₙ is as defined for formula (I),
to form a compound of formula 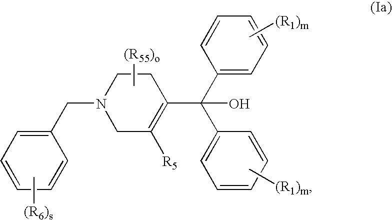
wherein R₁, R₅, R₅₅, R₆, m, o and s are as defined for formula (I); and where applicable, if desired,
(d) reacting the resulting compound of formula (Ia) with an oxidising agent to form a compound of formula (I) wherein q is 1.

The invention further relates to
(e) a process for the preparation of a compound of formula (I), or a salt thereof,
   wherein (R₁)ₘ and (R₂), are identical, R₅, R₅₅, R₆, o, q and s are as defined for formula (I) and R₃ and R₄ are hydrogen, which comprises reacting a compound of formula 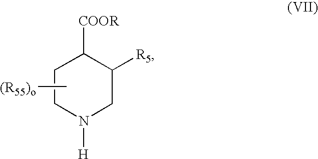
which is known or can be prepared by methods known per se and wherein R₅, R₅₅ and o are as defined for formula (I), in a manner analogous to the above Process Steps (a), (c) and (d).

The invention further relates to a process for the preparation of a compound of formula (I), or a salt thereof, wherein (R₁)ₘ and (R₂)ₙ are identical, R₅, R₅₅, R₆, o, q and s are as defined for formula (I) and R₃ and R₄ are hydrogen, which comprises
(f) reacting a compound of formula (VII), in a manner analogous to Process Step (a), with a compound of formula 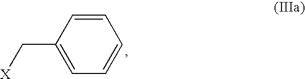
wherein X is a leaving group;
(g) reacting the resulting compound of formula 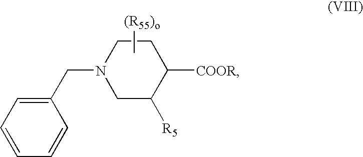
wherein R, R₅, R₅₅, o and R are as defined for formula (VII), in a manner analogous to Process Step (c), to form a compound of formula 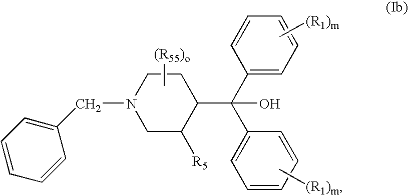
wherein (R₁)ₘ, (R₂)ₙ, R₅, R₅₅ and o are as defined for formula (I); and, where applicable,
(h) for the preparation of a compound of formula (I) wherein s is not 0, removing the benzyl group and further reacting the resulting compound of formula 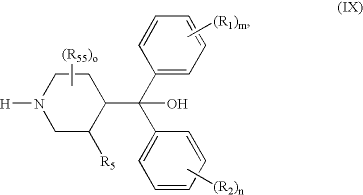
wherein (R₁)ₘ, R₅, R₅₅ and o are as defined for formula (I), analogously to Process Step (a) and, where applicable, (d).

(i) A process for the preparation of a compound of formula (VII), or a salt thereof, which comprises converting a compound of formula 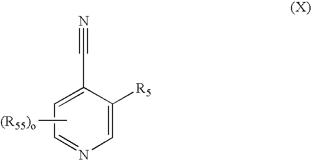
in the presence of an acid and an alcohol of formula HO-C₁-C₈alkyl into a compound of formula (II), or a salt thereof;
(k) converting that compound of formula (II) by hydrogenation into a compound of formula 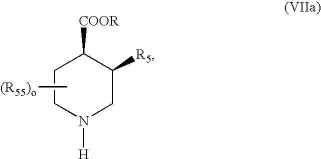
and, where applicable, if desired,
(I) transisomerising the compound of formula (VIIa) to form a compound of formula

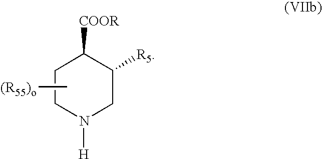

The invention further relates to a process for the preparation of a compound of formula (I) as defined above and wherein R₃ and R₄ together form a bond, which comprises
(m) reacting a compound of formula 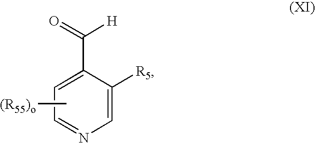
wherein R₅, R₅₅ and o are as defined for formula (1), in a manner analogous to Process Step (c), with a compound of formula (VI) to form a compound of formula 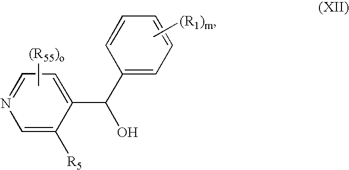
wherein (R₁)ₘ, R₅, R₅₅ and o are as defined for formula (I);
(n) oxidising the resulting compound of formula (XII) to form a compound of formula 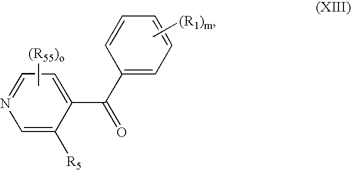
wherein (R₁)ₘ, R₅, R₅₅ and o are as defined for formula (I);
(o) reacting the compound of formula (XIII) in a manner analogous to Process Steps (c) and (m) to form a compound of formula 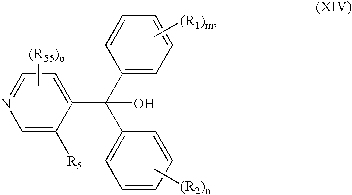
wherein (R₁)ₘ, R₅, R₅₅ and o are as defined for formula (I);
and further reacting the resulting compound of formula (XIV) in succession analogously to Process Steps (b), (a) and—if desired—(d).

The invention further relates to a process for the preparation of a compound of formula (VIII) as defined above, which comprises
(p) reacting a compound of the formula 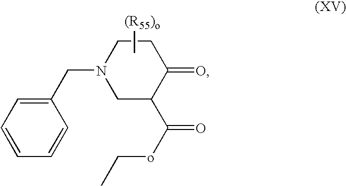
which is known or which can be prepared according to methods known per se, and wherein R₅₅ and o have the same meanings as defined under formula (I), in the presence of a base such as K₂CO₃ and a solvent such as acetone, with a compound of the fomula R₅—X, wherein R₅ is as defined under formula (I), and X is a leaving group, preferably Cl or Br,
(q) further treating the compound thus obtained of the formula 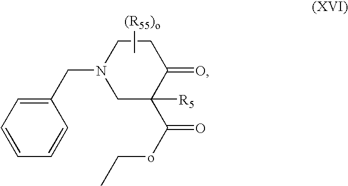
wherein R₅, R₅₅ and o have the same meanings as defined under formula (I), with an acid such as sulfuric acid,
(r) reacting the compound thus obtained of the formula 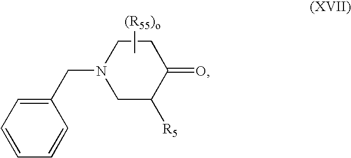
wherein R₅, R₅₅ and o have the same meanings as defined under formula (I), preferably in the presence of a base such as KO-tert-butyl, with TOSMIC of the formula 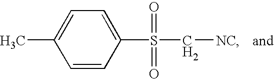
(s) treating the compound of the formula 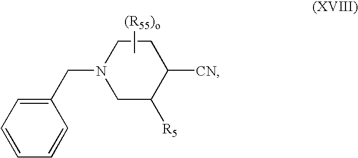
thus obtained, wherein R₅, R₅₅ and o have the same meanings as defined under formula (I), in the presence of an alcohol of the formula HO-C₁-C₈alkyl with a acid such as sulphuric acid.

The starting materials of formulae (III), (VI), (X) and (XV) mentioned hereinbefore and hereinafter, which are used for the preparation of the compounds of formula (I) in free form or in salt form, are known or can be prepared by methods known per se. Some of the compounds of formulae (II), (IV), (V), (VII), (IX) to (XIV) and (XVI) to (XVIII) are novel. The invention relates to them also.

The remarks made above regarding tautomers of compounds of formula (I) apply analogously to the starting materials mentioned hereinbefore and hereinafter with regard to their tautomers.

The reactions described hereinbefore and hereinafter are carried out in a manner known per se, for example in the absence or, customarily, in the presence of a suitable solvent or diluent or of a mixture thereof, the reactions being carried out, as required, with cooling, at room temperature or with heating, for example in a temperature range of approximately from −80° C. to the boiling temperature of the reaction medium, preferably from approximately 0° C. to approximately +150° C., and, if necessary, in a closed vessel, under pressure, under an inert gas atmosphere and/or under anhydrous conditions. Especially advantageous reaction conditions can be found in the Examples.

The reaction time is not critical; a reaction time of from approximately 0.1 to approximately 72 hours, especially from approximately 0.5 to approximately 24 hours, is preferred.

The product is isolated by customary methods, for example by filtration, crystallisation, distillation or chromatography, or any suitable combination of such methods.

Hereinbefore and hereinafter a leaving group is understood as being any removable group known to the person skilled in the art that normally comes into consideration in chemical reactions, especially halogens, such as fluorine, chlorine, bromine, iodine, —O—C(═O)-A, —O—P(═O)(W)₂, —O—Si(C₁-C₈alkyl)₃, —O—(C₁-C₈alkyl), —O-aryl, —O—S(═O)₂W, —S—P(═O)(W)₂, —S—P(═S)(W)₂, —S—S—(C₁-C₈alkyl), —S—S-aryl, —S-(C₁-C₈alkyl), —S-aryl, —S(═O)W or —S(═O)₂W, wherein W is unsubstituted or substituted C₁-C₈alkyl, C₂-C₈alkenyl, C₂-C₈-alkynyl, unsubstituted or substituted aryl, unsubstituted or substituted benzyl, C₁-C₈alkoxy or di(C₁-C₈alkyl)amine wherein the alkyl groups are independent of each other; NO₃, NO₂, or sulfate, sulfite, phosphate, phosphite, carboxylate, imino ester, N₂ or carbamate. Especially preferred as the leaving group are chlorine and bromine, more especially chlorine.

The starting materials mentioned hereinbefore and hereinafter that are used for the preparation of the compounds of formula (I) and, where applicable, their tautomers are known or can be prepared by methods known per se, for example as indicated below.

In the reaction according to Process Variants (a) and (f), inert solvents are used, such as, for example, benzene, toluene, xylenes, acetonitrile, propionitrile, ethyl acetate, propyl acetate, butyl acetate, acetone, diethyl ketone, methyl ethyl ketone or methyl isobutyl ketone. The temperature range is from room temperature to the reflux temperature of the solvent concerned, the reflux temperature being preferred.

Process Variant (b): The reaction is preferably carried out in alcohols, such as, for example, methanol or ethanol, in a temperature range of from 0° C. to +50° C., preferably at room temperature. A preferred reducing agent is sodium borohydride.

Process Variants (c), (q), (m), (o): Dialkyl ethers or tetrahydrofuran are preferably used as solvent; the reactions are carried out in a temperature range of from −70° C. to room temperature, and magnesium or n-butyllithium is used as metallating agent.

In Process Variant (d), alcohols, such as, for example, methanol or ethanol, are preferably used as solvent. The procedure is preferably carried out at room temperature. The oxidising agents used are, for example, inorganic peroxides, such as sodium perborate, potassium permanganate or hydrogen peroxide; or organic peracids, such as perbenzoic acid, m-chloro-perbenzoic acid (mCPBA) or peracetic acid; or mixtures of organic acids and hydrogen peroxide, such as, for example, acetic acid/hydrogen peroxide. Especially suitable are H₂O₂ or peracids, more especially H₂O₂.

Process Variant (h): The reaction is preferably carried out in alcohols such as methanol or ethanol, in a temperature range of from 0 to 50° C., especially at room temperature, under normal pressure and using a catalyst, especially palladium-on-carbon.

Process Variant (i): The hydrolysis is carried out using a mineral acid, especially hydrochloric acid or sulfuric acid, at from 20 to 150° C., and the subsequent esterification is usually carried out with a lower alcohol, such as methanol or ethanol.

Process Variant (k): The reduction is effected with the use of a catalyst, such as, for example, PtO₂ or palladium-on-carbon, in a solvent, such as, for example, an alcohol, or in an organic acid, for example acetic acid, with hydrogen under normal pressure or also at elevated pressure, preferably from 1 to 10 atm.

Process Variant (I): The isomerisation is effected with the use of bases, especially alcoholates, such as, for example, sodium methanolate, in an organic solvent, such as, for example, an alcohol, for example methanol, and between room temperature and the boiling point of the solvent concerned.

Process Variant (n): The process is preferably carried out in an inert solvent such as a hydrocarbon or a halogenated hydrocarbon, such as, for example, methylene chloride, and with dimethyl sulfoxide/oxalyl chloride or pyridinium dichromate as oxidising agent.

Compounds of formula (I) obtainable in accordance with the process or by other methods can be converted in a manner known per se into other compounds of formula (I) by replacing one or more of the substituents of the starting compound of formula (I) in a conventional manner by one (or more) other substituent(s) according to the invention.

In so doing, depending upon the choice of reaction conditions and starting materials suitable for the respective purpose, it is possible for only one substituent to be replaced by a different substituent according to the invention in one reaction step or for several substituents to be replaced by different substituents according to the invention in the same reaction step.

Salts of compounds of formula (I) may be prepared in a manner known per se. Salts of compounds of formula (I) with bases are obtained, for example, by treating the free compounds with a suitable base or with a suitable ion-exchange reagent.

Salts of compounds of formula (I) may be converted in a conventional manner into the free compounds of formula (I), for example by treatment with a suitable acid or with a suitable ion-exchange reagent.

Salts of compounds of formula (I) may be converted in a manner known per se into other salts of a compound of formula (I).

The compounds of formula (I) in free form or in salt form may be in the form of one of the possible isomers or in the form of a mixture thereof, for example according to the number and the absolute and relative configuration of asymmetric carbon atoms occurring in the molecule and/or according to the configuration of non-aromatic double bonds occurring in the molecule, they may be in the form of pure isomers, such as antipodes and/or diastereoisomers, or in the form of mixtures of isomers, such as mixtures of enantiomers, for example racemates, mixtures of diastereoisomers or mixtures of racemates. The invention relates both to the pure isomers and to all possible mixtures of isomers and is to be interpreted accordingly hereinbefore and hereinafter, even if stereochemical details are not mentioned specifically in every case.

Mixtures of diastereoisomers, mixtures of racemates and mixtures of double-bond isomers of compounds of formula (I), in free form or in salt form, that are obtainable in accordance with the process depending upon the starting materials and procedures chosen, or by other means, can be separated into the pure diastereoisomers or racemates in known manner on the basis of the physico-chemical differences between the constituents, for example by fractional crystallisation, distillation and/or chromatography.

Correspondingly obtainable mixtures of enantiomers, such as racemates, can be separated into the optical antipodes by known methods, for example by recrystallisation from an optically active solvent, by chromatography on chiral adsorbents, for example high-pressure liquid chromatography (HPLC) on acetyl cellulose, with the aid of suitable micro-organisms, by cleavage with specific, immobilised enzymes, or via the formation of inclusion compounds, for example using chiral crown ethers, only one enantiomer being complexed, or by conversion into diastereoisomeric salts and separation of the resulting mixture of diastereoisomers, for example on the basis of their different solubilities, by fractional crystallisation, into the diastereoisomers from which the desired enantiomer can be freed by the action of suitable agents.

Apart from by separation of corresponding mixtures of isomers, pure diastereoisomers and enantiomers can be obtained according to the invention also by generally known methods of diastereoselective synthesis and enantioselective synthesis, respectively, for example by carrying out the process according to the invention using starting materials having correspondingly suitable stereochemistry.

In each case it is advantageous to isolate or synthesise the biologically more active isomer, e.g. enantiomer or diastereoisomer, or mixture of isomers, e.g. mixture of enantiomers or mixture of diastereoisomers, where the individual components have differing biological activity.

The compounds of formula (I) in free form or in salt form may also be obtained in the form of their hydrates and/or may include other solvents, for example solvents which may have been used for the crystallisation of compounds in solid form.

The invention relates to all those embodiments of the process according to which a compound obtainable as starting material or intermediate at any stage of the process is used as starting material and some or all of the remaining steps are carried out or a starting material is used in the form of a derivative or salt and/or its racemates or antipodes or, especially, is formed under the reaction conditions.

In the process of the present invention it is preferable to use those starting materials and intermediates, each in free form or in salt form, which result in the compounds of formula (I), or salts thereof, described at the beginning as being especially valuable.

The invention relates especially to the preparation processes described in Examples P1 and P2.

In the area of pest control, the compounds of formula (I) according to the invention are active ingredients exhibiting valuable preventive and/or curative activity with a very advantageous biocidal spectrum and a very broad spectrum, even at low rates of concentration, while being well tolerated by warm-blooded animals, fish and plants. They are, surprisingly, equally suitable for controlling both plant pests and ecto- and endo-parasites in humans and more especially in productive livestock, domestic animals and pets. They are effective against all or individual development stages of normally sensitive animal pests, but also of resistant animal pests, such as insects and members of the order Acarina, nematodes, cestodes and trematodes, while at the same time protecting useful organisms. The insecticidal or acaricidal activity of the active ingredients according to the invention may manifest itself directly, i.e. in the mortality of the pests, which occurs immediately or only after some time, for example during moulting, or indirectly, for example in reduced oviposition and/or hatching rate, good activity corresponding to a mortality of at least 50 to 60%.

The action of the compounds according to the invention and the compositions comprising them against animal pests can be significantly broadened and adapted to the given circumstances by the addition of other insecticides, acaricides or nematicides. Suitable additives include, for example, members of the following classes of active ingredient: organophosphorus compounds, nitrophenols and derivatives, formamidines, ureas, carbamates, pyrethroids, chlorinated hydrocarbons and Bacillus thuringiensis preparations.

Examples of especially suitable mixing partners include: azamethiphos; chlorfenvinphos; bupirimate; cypermethrin, cypermethrin high-cis; cyromazine; diafenthiuron; diazinon; dichlorvos; dicrotophos; dicyclanil; fenoxycarb; fluazuron; furathiocarb; isazofos; iodfenphos; kinoprene; lufenuron; methacriphos; methidathion; monocrotophos; phosphamidon; profenofos; diofenolan; a compound obtainable from the Bacillus thuringiensis strain GC91 or from NCTC11821; pymetrozine; bromopropylate; methoprene; disulfoton; quinalphos; taufluvalinate; thiocyclam; thiometon; aldicarb; azinphos-methyl; benfuracarb; bifenthrin; buprofezin; carbofuran; dibutylaminothio; cartap; chlorfluazuron; chlorpyrifos; cyfluthrin; alpha-cypermethrin; zeta-cypermethrin; deltamethrin; diflubenzuron; endosulfan; ethiofencarb; fenitrothion; fenazaquin; fenobucarb; fenvalerate; formothion; methiocarb; heptenophos; imidacloprid; isoprocarb; methamidophos; methomyl; mevinphos; parathion; parathionmethyl; phosalone; pirimicarb; propoxur; teflubenzuron; terbufos; triazamate; abamectin; fenobucarb; tebufenozide; fipronil; beta-cyfluthrin; silafluofen; fenpyroximate; pyridaben; primicarb; pyriproxyfen; pyrimidifen; nemathorin; nitenpyram; NI-25; acetamiprid; avermectin B₁ (abamectin); a plant extract that is active against insects; a preparation comprising nematodes that are active against insects; a preparation obtainable from Bacillus subtilis; a preparation comprising fungithat are active against insects; a preparation comprising viruses that are active against insects; AC 303 630; acephate; acrinathrin; alanycarb; alphamethrin; amitraz; AZ 60541; azinphos A; azinphos M; azocyclotin; bendiocarb; bensultap; beta-cyfluthrin; BPMC; brofenprox; bromophos A; bufencarb; butocarboxin; butyl-pyridaben; cadusafos; carbaryl; carbophenothion; chloethocarb; chlorethoxyfos; chlormephos; cis-resmethrin; clocythrin; clofentezine; cyanophos; cycloprothrin; cyhexatin; demeton M; demeton S; demeton-S-methyl; dichlofenthion; dicliphos; diethion; dimethoate; dimethylvinphos; dioxathion; edifenphos; emamectin; esfenvalerate; ethion; ethofenprox; ethoprophos; etrimphos; fenamiphos; fenbutatin oxide; fenothiocarb; fenpropathrin; fenpyrad; fenthion; fluazinam; flucycloxuron; flucythrinate; flufenoxuron; flufenprox; fonophos; fosthiazate; fubfenprox; HCH; hexaflumuron; hexythiazox; IKI-220; iprobenfos; isofenphos; isoxathion; ivermectin; lambda-cyhalothrin; malathion; mecarbam; mesulfenphos; metaldehyde; metolcarb; milbemectin; moxidectin; naled; NC 184; omethoate; oxamyl; oxydemethon M; oxydeprofos; permethrin; phenthoate; phorate; phosmet; phoxim; pirimiphos M; pirimiphos A; promecarb; propaphos; prothiofos; prothoate; pyrachlophos; pyradaphenthion; pyresmethrin; pyrethrum; RH 5992; salithion; sebufos; sulfotep; sulprofos; tebufenpyrad; tebupirimphos; tefluthrin; temephos; terbam; tetrachlorvinphos; thiacloprid; thiamethoxam; thiafenox; thiodicarb; thiofanox; thionazin; thuringiensin; tralomethrin; triarthene; triazophos; triazuron; trichlorfon; triflumuron; trimethacarb; vamidothion; xylylcarb; YI 5301/5302; zetamethrin; DPX-MP062; RH-2485; D2341 or XMC (3,5-xylyl methylcarbamate).

The said animal pests include, for example, those mentioned in European Patent Application EP-A-736 252, page 5, line 55, to page 6, line 55. The pests mentioned therein are therefore included by reference in the subject matter of the present invention.

It is also possible to control pests of the class Nematoda using the compounds according to the invention. Such pests include, for example, root knot nematodes, cyst-forming nematodes and also stem and leaf nematodes;
especially of Heterodera spp., e.g. Heterodera schachtii, Heterodora avenae and Heterodora trifolii; Globodera spp., e.g. Globodera rostochiensis; Meloidogyne spp., e.g. Meloidogyne incognita and Meloidogyne javanica; Radopholus spp., e.g. Radopholus similis; Pratylenchus, e.g. Pratylenchus neglectans and Pratylenchus penetrans; Tylenchulus, e.g. Tylenchulus semipenetrans; Longidorus, Trichodorus, Xiphinema, Ditylenchus, Apheenchoides and Anguina; especially Meloidogyne, e.g. Meloidogyne incognita, and Heterodera, e.g. Heterodera glycines.

An especially important aspect of the present invention is the use of the compounds of formula (I) according to the invention in the protection of plants against parasitic feeding pests.

The compounds according to the invention can be used to control, i.e. to inhibit or destroy, pests of the mentioned type occurring on plants, especially on useful plants and ornamentals in agriculture, in horticulture and in forestry, or on parts of such plants, such as the fruits, blossoms, leaves, stems, tubers or roots, while in some cases plant parts that grow later are still protected against those pests.

Target crops include especially cereals, such as wheat, barley, rye, oats, rice, maize and sorghum; beet, such as sugar beet and fodder beet; fruit, e.g. pomes, stone fruit and soft fruit, such as apples, pears, plums, peaches, almonds, cherries and berries, e.g. strawberries, raspberries and blackberries; leguminous plants, such as beans, lentils, peas and soybeans; oil plants, such as rape, mustard, poppy, olives, sunflowers, coconut, castor oil, cocoa and groundnuts; cucurbitaceae, such as marrows, cucumbers and melons; fibre plants, such as cotton, flax, hemp and jute; citrus fruits, such as oranges, lemons, grapefruit and mandarins; vegetables, such as spinach, lettuce, asparagus, brassicas, carrots, onions, tomatoes, potatoes and paprika; lauraceae, such as avocado, cinnamon and camphor; and tobacco, nuts, coffee, aubergines, sugar cane, tea, pepper, vines, hops, bananas, natural rubber plants and ornamentals.

Further areas of use of the compounds according to the invention are the protection of stored goods and storerooms and the protection of raw materials, and also in the hygiene sector, especially the protection of domestic animals and productive livestock against pests of the mentioned type, more especially the protection of domestic animals, especially cats and dogs, from infestation by fleas, ticks and nematodes.

The invention therefore relates also to pesticidal compositions, such as emulsifiable concentrates, suspension concentrates, directly sprayable or dilutable solutions, spreadable pastes, dilute emulsions, wettable powders, soluble powders, dispersible powders, wettable powders, dusts, granules and encapsulations in polymer substances, that comprise at least one of the compounds according to the invention, the choice of formulation being made in accordance with the intended objectives and the prevailing circumstances.

The active ingredient is used in those compositions in pure form, a solid active ingredient, for example, in a specific particle size, or preferably together with at least one of the adjuvants customary in formulation technology, such as extenders, e.g. solvents or solid carriers, or surface-active compounds (surfactants). In the area of parasite control in humans, domestic animals, productive livestock and pets it will be self-evident that only physiologically tolerable additives are used.

As formulation adjuvants there are used, for example, solid carriers, solvents, stabilisers, “slow release” adjuvants, colourings and optionally surface-active substances (surfactants). Suitable carriers and adjuvants include all substances customarily used. As adjuvants, such as solvents, solid carriers, surface-active compounds, non-ionic surfactants, cationic surfactants, anionic surfactants and further adjuvants in the compositions used according to the invention, there come into consideration, for example, those described in EP-A-736 252, page 7, line 51 to page 8, line 39.

The compositions for use in crop protection and in humans, domestic animals and productive livestock generally comprise from 0.1 to 99%, especially from 0.1 to 95%, of active ingredient and from 1 to 99.9%, especially from 5 to 99.9%, of at least one solid or liquid adjuvant, the composition generally including from 0 to 25%, especially from 0.1 to 20%, of surfactants (%=% by weight in each case). Whereas commercial products will preferably be formulated as concentrates, the end user will normally employ dilute formulations having considerably lower concentrations of active ingredient.

Preferred crop protection products have especially the following compositions (%=percent by weight):

| | |
|---|---|
| Emulsifiable concentrates: | |
| active ingredient: | 1 to 90%, preferably 5 to 20% |
| surfactant: | 1 to 30%, preferably 10 to 20% |
| solvent: | 5 to 98%, preferably 70 to 85% |
| Dusts: | |
| active ingredient: | 0.1 to 10%, preferably 0.1 to 1% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surfactant: | 1 to 40%, preferably 2 to 30% |
| Wettable powders: | |
| active ingredient: | 0.5 to 90%, preferably 1 to 80% |
| surfactant: | 0.5 to 20%, preferably 1 to 15% |
| solid carrier: | 5 to 99%, preferably 15 to 98% |
| Granules: | |
| active ingredient: | 0.5 to 30%, preferably 3 to 15% |
| solid carrier: | 99.5 to 70%, preferably 97 to 85% |

The compositions according to the invention may also comprise further solid or liquid adjuvants, such as stabilisers, e.g. vegetable oils or epoxidised vegetable oils (e.g. epoxidised coconut oil, rapeseed oil or soybean oil), antifoams, e.g. silicone oil, preservatives, viscosity regulators, binders and/or tackifiers as well as fertilisers or other active ingredients for obtaining special effects, e.g. acaricides, bactericides, fungicides, nematicides, molluscicides or selective herbicides.

The crop protection products according to the invention are prepared in known manner, in the absence of adjuvants, e.g. by grinding, sieving and/or compressing a solid active ingredient or mixture of active ingredients, for example to a certain particle size, and in the presence of at least one adjuvant, for example by intimately mixing and/or grinding the active ingredient or mixture of active ingredients with the adjuvant(s). The invention relates likewise to those processes for the preparation of the compositions according to the invention and to the use of the compounds of formula (I) in the preparation of those compositions.

The invention relates also to the methods of application of the crop protection products, i.e. the methods of controlling pests of the mentioned type, such as spraying, atomising, dusting, coating, dressing, scattering or pouring, which are selected in accordance with the intended objectives and the prevailing circumstances, and to the use of the compositions for controlling pests of the mentioned type. Typical rates of concentration are from 0.1 to 1000 ppm, preferably from 0.1 to 500 ppm, of active ingredient. The rates of application per hectare are generally from 1 to 2000 g of active ingredient per hectare, especially from 10 to 1000 g/ha, preferably from 20 to 600 g/ha.

A preferred method of application in the area of crop protection is application to the foliage of the plants (foliar application), the frequency and the rate of application being dependent upon the risk of infestation by the pest in question. However, the active ingredient can also penetrate the plants through the roots (systemic action) when the locus of the plants is impregnated with a liquid formulation or when the active ingredient is incorporated in solid form into the locus of the plants, for example into the soil, e.g. in granular form (soil application). In the case of paddy rice crops, such granules may be applied in metered amounts to the flooded rice field.

The crop protection products according to the invention are also suitable for protecting plant propagation material, e.g. seed, such as fruits, tubers or grains, or plant cuttings, against animal pests. The propagation material can be treated with the composition before planting: seed, for example, can be dressed before being sown. The active ingredients according to the invention can also be applied to grains (coating), either by impregnating the seeds in a liquid formulation or by coating them with a solid formulation. The composition can also be applied to the planting site when the propagation material is being planted, for example to the seed furrow during sowing. The invention relates also to such methods of treating plant propagation material and to the plant propagation material so treated.

The following Examples serve to illustrate the invention. They do not limit the invention. Temperatures are given in degrees Celsius and mixing ratios of solvents are given in parts by volume.

### PREPARATION EXAMPLES

P1: Preparation of the Compound of Formula

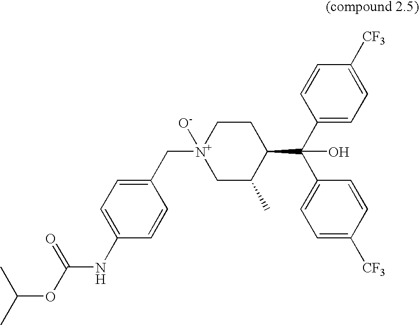

P1.1: Preparation of the Compound of Formula

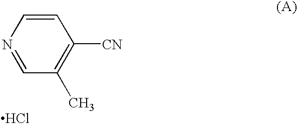

60 g of 3-picoline N-oxide and 60 ml of ethyl iodide are mixed with stirring during which the temperature is maintained below 40° C. The reaction mixture is left to stand for 16 hours. Then 600 ml of water are added and the aqueous phase is extracted twice with 300 ml of diethyl ether. 70 g of potassium cyanide in 180 ml of water are added dropwise to the aqueous phase, with stirring, over a period of 1 hour at 50° C. Stirring is then carried out for one hour, followed by cooling and extraction twice with 300 ml of diethyl ether. The combined ethereal phases are washed with saturated sodium chloride solution, dried over sodium sulfate and concentrated. The crude product is distilled at 20 mbar at 100-110° C. 280 ml of methanol are saturated with HCl gas, the distillate is added and boiling is carried out for 7 hours. That solution is cooled to approximately 5° C., and the product which precipitates is removed by filtration and dried, yielding compound (A) having a melting point of 234-237° C.

P1.2: Preparation of the Compound of Formula

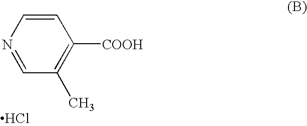

3 g of compound (A) are added to 30 ml of concentrated hydrochloric acid and the mixture is stirred for 16 hours at 110° C. The mixture is concentrated by evaporation and the residue is dried in vacuo, yielding compound (B).

P1.3: Preparation of the Compound of Formula

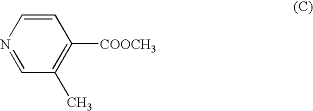

190 ml of methanol are placed in a reaction vessel and HCl gas is introduced, with ice-cooling, until saturation is reached. Then, at 0° C., 41.6 g of compound (B) are added and the mixture is stirred under reflux for 4 hours. The reaction mixture is concentrated to dryness, and the residue is dissolved in 190 ml of water, rendered alkaline with sodium hydrogen carbonate and extracted three times with 100 ml of diethyl ether each time. The combined ethereal phases are dried over sodium sulfate and then evaporated to dryness, yielding the compound of formula (C).

P1.4: Preparation of the Compound of Formula

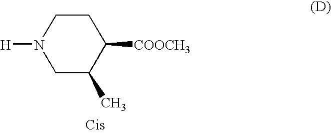

25.8 g of compound (C) are introduced into 200 ml of diethyl ether, followed by cooling to 5° C. HCl gas is then introduced until saturation is reached, and the temperature is maintained at 110C. The mixture is filtered and the residue is washed with a small quantity of diethyl ether and dried in vacuo. The resulting hydrochloride of compound (C) is hydrogenated in 290 ml of acetic acid and 3 g of platinum oxide for 12 hours at 4 bar and at room temperature. After filtration of the reaction mixture, concentration is carried out, and the residue is taken up in 150 ml of water, adjusted to pH 11 with potassium carbonate and extracted 4 times with approximately 200 ml of methylene chloride. The methylene chloride phases are combined, dried over sodium sulfate, filtered and concentrated by evaporation, yielding the desired product (D) in the form of an oil.

P1.5: Preparation of the Compound of Formula

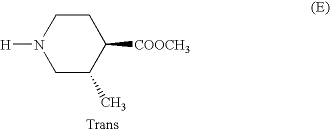

3.2 g of sodium are added to 350 ml of methanol and then 21.5 g of compound (D) are added. The mixture is stirred at reflux temperature for 48 hours, cooled, acidified with acetic acid and concentrated in vacuo. The residue is stirred with 700 ml of methylene chloride and 1 litre of saturated potassium carbonate solution, and the aqueous phase is separated and extracted once with 200 ml of methylene chloride. The combined methylene chloride phases are washed with sodium chloride solution, dried (sodium sulfate), concentrated, and dried under a high vacuum, yielding compound (E) in the form of a yellow oil.

P1.6: Preparation of the Compound of Formula

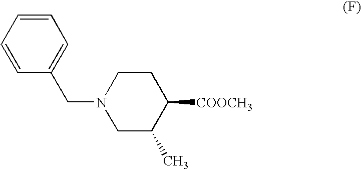

5 g of the compound of formula (E) are introduced into 80 ml of dimethyl sulfoxide. Then, at room temperature, 5.5 g of benzyl bromide and then 15 g of diisopropylethylamine are added dropwise and the mixture is stirred at room temperature for 16 hours. The reaction mixture is poured onto 200 ml of saturated potassium carbonate solution and washed twice with 200 ml of ethyl acetate each time. The combined organic phases are washed twice with water and once with saturated sodium chloride solution and are dried over sodium sulfate. The solvent is then removed by evaporation. The residue is purified on silica gel using ethyl acetate/hexane (1:3) as eluant, yielding the compound of formula (F).

P1.7: Preparation of the Compound of Formula

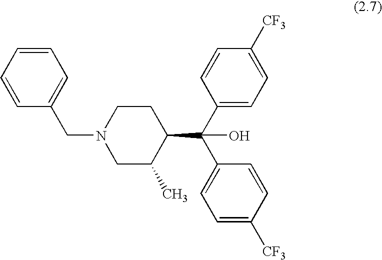

6.8 g of 4-bromobenzotrifluoride are introduced into 40 ml of diethyl ether under nitrogen. At 5° C., 19 ml of a 1.6-molar n-butyllithium solution in hexane are added. Then, 2.5 g of compound (F) are added dropwise at 5° C. and stirring is continued for one hour at room temperature and for 2 hours at 45° C. The mixture is cooled, 60 ml of acetic acid (10% in water) are added and the mixture is extracted with ethyl acetate. The organic phase is washed with water and potassium carbonate solution and dried over sodium sulfate, and the solvent is removed by evaporation. Compound 2.7 is obtained after purification on silica gel using ethyl acetate/hexane (1:1) as eluant.

P1.8: Preparation of the Compound of Formula

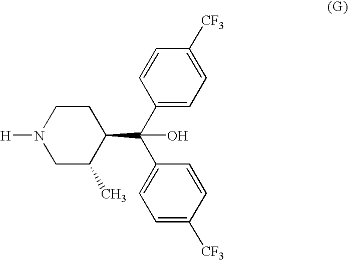

4 g of compound (2.7) are hydrogenated in 40 ml of methanol and 1.2 g of palladium-on-carbon (5% Pd on carbon) at room temperature under normal pressure. The reaction mixture is filtered, concentrated in vacuo and dried under a high vacuum, yielding compound (G) in the form of a resin.

P1.9: Preparation of the Compound of Formulae

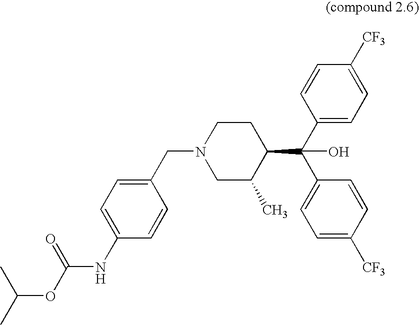

To 2.9 g of compound (G) in 17 ml of dimethyl sulfoxide there are added first 1.64 g of the compound of formula 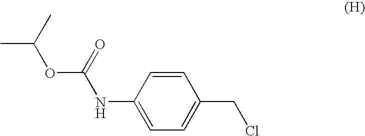
and then 3.6 g of diisopropylethylamine. The reaction mixture is stirred for 3 hours at room temperature, poured onto saturated aqueous potassium carbonate solution and extracted twice with ethyl acetate, and the ethyl acetate phase is washed with water and dried over sodium sulfate. The ethyl acetate is removed by evaporation. Purification of the residue on silica gel using ethyl acetate/hexane (2:1) as eluant yields the desired product having a melting point of 66-67° C. (Compound 2.6).

P1.10: Preparation of the Title Compound

1.2 g of compound (2.6) are stirred in 40 ml of methanol and 6.7 g of hydrogen peroxide (30% in water) for 24 hours at a bath temperature of 55° C. The reaction mixture is concentrated, dissolved in ethyl acetate, washed twice with water and once with saturated sodium chloride solution, dried over sodium sulfate and concentrated. The residue is stirred thoroughly with a small quantity of ethyl acetate, and the product which precipitates is removed by filtration and washed with hexane, yielding the title compound having a melting point of 188-190° C. (compound 2.5).

P2: Preparation of the Compound of Formula

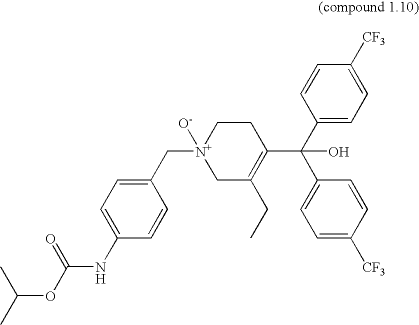

P2.1: Preparation of the Compound of Formula

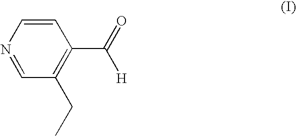

A solution of 34.3 ml of ethylmagnesium chloride in 50 ml of tetrahydrofuran is added dropwise at −70° C. to 7.3 g of indium trichloride in 200 ml of tetrahydrofuran and, after stirring for 30 minutes, the reaction temperature is allowed to rise slowly to room temperature. That solution is added to a solution of 14.9 g of 3-bromo-4-pyridine carbaldehyde and 2.8 g of PdCl₂ (PPh₃)₂ in 240 ml of tetrahydrofuran and the reaction mixture is heated under reflux for 20 hours. 5 ml of methanol are then added and the mixture is concentrated in vacuo, stirred thoroughly with diethyl ether, filtered off and concentrated in vacuo once more. The residue is chromatographed on silica gel using ethyl acetate/hexane (1:1), yielding 3-ethyl-4-pyridine carbaldehyde (I) in the form of a yellow oil.

P2.2: Preparation of the compound of formula

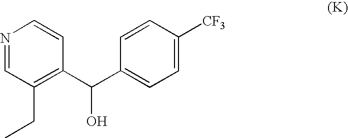

To a solution of 5.76 ml of 4-bromo-benzotrifluoride in 120 ml of tetrahydrofuran there are added dropwise at −70° C. 26.1 ml of n-butyllithium (1.6M in hexane) and, after stirring for 10 minutes, 5.12 g of compound (I). After 1 hour at −70° C., the reaction temperature is allowed to rise slowly to room temperature. 60 ml of 5% acetic acid are added dropwise, dilution is carried out with 100 ml of tert-butyl methyl ether, the aqueous phase is separated and the organic phase is washed with sodium chloride solution, dried and concentrated in vacuo. The residue is chromatographed on silica gel using ethyl acetate/hexane (3:1), yielding compound (K) in the form of a yellow oil.

P2.3: Preparation of the compound of formula

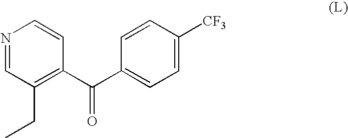

To 2.42 ml of oxalyl chloride in 80 ml of dichloromethane there are added dropwise at −70° C. 4.28 ml of dimethyl sulfoxide in 60 ml of dichloromethane and, after stirring for 30 minutes, 6.95 g of compound (K) in 40 ml of dichloromethane. After 1 hour, 17 ml of triethylamine in 30 ml of dichloromethane are added dropwise and the reaction temperature is allow to rise slowly to room temperature. The reaction mixture is poured onto 100 ml of ice-water, and the organic phase is separated, dried, and concentrated in vacuo. The residue is chromatographed on silica gel using ethyl acetate/hexane (1:1), yielding compound (L) in the form of a yellow oil.

P2.4: Preparation of the Compound of Formula

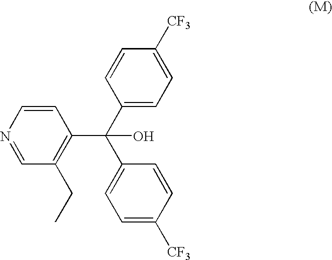

To a solution of 3.45 ml of 4-bromo-benzotrifluoride in 100 ml of tetrahydrofuran there are added dropwise at −70° C. 15.6 ml of n-butyllithium (1.6M in hexane) and, after stirring for 10 minutes, 5.91 g of compound (L). The reaction temperature is then allowed to rise slowly to room temperature. 40 ml of acetic acid (5%) are added dropwise, dilution is carried out with 100 ml of tert-butyl methyl ether, the aqueous phase is separated and the organic phase is washed with aqueous sodium chloride solution, dried, and concentrated in vacuo. Recrystallisation from dichloromethane/hexane yields compound (M) in the form of colour-less crystals, m.p. 202-203° C.

P2.5: Preparation of the compound of formula

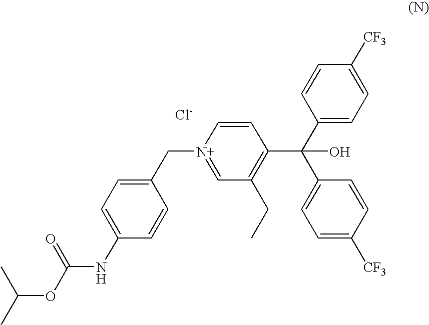

2.0 g of compound (M) and 1.18 g of compound (H) from Example P1.9 are heated under reflux in 20 ml of nitromethane for 14 hours. The solvent is then distilled oft in vacuo. Compound (N) is obtained in the form of beige crystals, m.p. 214-220° C., from dichloromethane/diethyl ether.

P2.6: Preparation of Compound 1.10

0.28 g of sodium borohydride is added in portions to 2.51 g of compound (N) in 25 ml of methanol and stirring is carried out for 20 minutes. After the addition of 1 ml of acetone, concentration is carried out in vacuo, ethyl acetate is added and the mixture is washed once with water and once with sodium chloride solution, dried, and concentrated in vacuo. Chromatography on silica gel using ethyl acetate/hexane (1:3) yields compound 1.10 in the form of a foam.
g) Preparation of the Title Compound

0.92 g of compound (1.10) in 15 ml of methanol are stirred with 3.8 ml of hydrogen peroxide (30% in water) at 50° C. for 24 hours. The solvent is distilled off in vacuo, ethyl acetate is added to the residue, followed by washing once with water and once with sodium chloride solution, drying, and concentration in vacuo. The title compound is obtained in the form of colourless crystals, m.p. 207-211° C. (compound 1.11), from dichloromethane/diethyl ether/hexane.

Example P3: The further compounds of Tables 1 and 2 can also be prepared in a manner analogous to that described above. In the Tables, m.p. is the melting point in ° C.; in the column m.p. other physical properties are given as well. Me is methyl, Et is ethyl, prop is n-propyl, i-prop is isopropyl, i-but is isobutyl, c-prop is cyclopropyl and 2-ethyl-2H-tetrazole-5-yl is the substituent of the formula

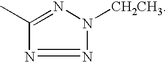

TABLE 1
Compounds of formula

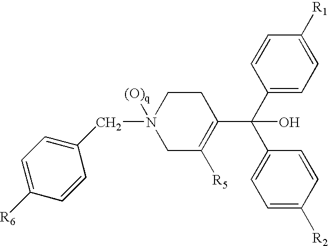

| No. | R₁ | R₂ | q | R₅ | R₆ | m.p. |
|---|---|---|---|---|---|---|
| 1.1 | CF₃ | CF₃ | 1 | CH₃ | 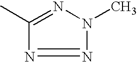 | 115-120 |
| 1.2 | CF₃ | CF₃ | 0 | CH₃ | 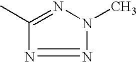 | resin |
| 1.3 | CF₃ | CF₃ | 0 | CH₃ | NHCOO-i-prop | 69-75 |
| 1.4 | CF₃ | CF₃ | 1 | CH₃ | NHCOO-i-prop | 105-110 |
| 1.5 | CF₃ | CF₃ | 0 | CH₃ | H | resin |
| 1.6 | CF₃ | CF₃ | 0 | CH₃ | NO₂ | amorphous |
| 1.7 | CF₃ | CF₃ | 0 | CH₃ | NH₂ | amorphous |
| 1.8 | CF₃ | CF₃ | 0 | CH₃ | NHCOO—CH₂— | amorphous |
| | | | | | C≡CH | |
| 1.9 | CF₃ | CF₃ | 1 | CH₃ | NHCOO—CH₂— | amorphous |
| | | | | | C≡CH | |
| 1.10 | CF₃ | CF₃ | 0 | C₂H₅ | NHCOO-i-prop | amorphous |
| 1.11 | CF₃ | CF₃ | 1 | C₂H₅ | NHCOO-i-prop | 207-211 |
| 1.12 | CF₃ | CF₃ | 0 | OCH₃ | 2-ethyl-2H-tetrazole- | 62-65 |
| | | | | | 5-yl | |
| 1.13 | CF₃ | CF₃ | 0 | OCH₃ | NHCOO-i-prop | 65-69 |
| 1.14 | OCF₃ | OCF₃ | 0 | CH₃ | NHCOO-i-prop | |
| 1.15 | OCF₃ | OCF₃ | 1 | CH₃ | NHCOO-i-prop | |
| 1.16 | OCF₃ | OCF₃ | 0 | CH₃ | 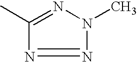 | |
| 1.17 | OCF₃ | OCF₃ | 1 | CH₃ | 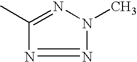 | |
| 1.18 | Cl | Cl | 0 | CH₃ | NHCOO-i-prop | |
| 1.19 | Cl | Cl | 1 | CH₃ | NHCOO-i-prop | |
| 1.20 | CF₃ | CF₃ | 0 | F | NHCOO-i-prop | amorphous |

TABLE 2
Compounds of formula

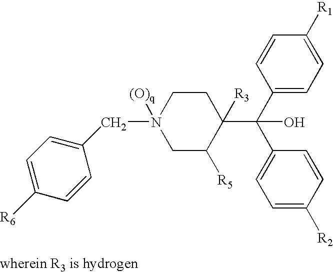

| No. | R₁ | R₂ | R₅ | R₃/R₅ | Q | R₆ | m.p. |
|---|---|---|---|---|---|---|---|
| 2.1 | CF₃ | CF₃ | CH₃ | cis | 0 | 2-ethyl-2H-tetrazole-5-yl | resin |
| 2.2 | CF₃ | CF₃ | CH₃ | trans | 0 | 2-ethyl-2H-tetrazole-5-yl | resin |
| 2.3 | CF₃ | CF₃ | CH₃ | cis | 1 | 2-ethyl-2H-tetrazole-5-yl | 177-179 |
| 2.4 | CF₃ | CF₃ | CH₃ | trans | 1 | 2-ethyl-2H-tetrazole-5-yl | 150-155 |
| 2.5 | CF₃ | CF₃ | CH₃ | cis | 1 | NHCOO-i-prop | 188-190 |
| 2.6 | CF₃ | CF₃ | CH₃ | cis | 0 | NHCOO-i-prop | 66-67 |
| 2.7 | CF₃ | CF₃ | CH₃ | cis | 0 | H | |
| 2.8 | CF₃ | CF₃ | OH | mixture | 0 | NHCOO-i-prop | resin |
| | | | | cis/trans | | | |
| 2.9 | CF₃ | CF₃ | OH | mixture | 1 | NHCOO-i-prop | resin |
| | | | | cis/trans | | | |
| 2.10 | CF₃ | CF₃ | OMe | mixture | 0 | NHCOO-i-prop | |
| | | | | cis/trans | | | |
| 2.11 | CF₃ | CF₃ | OMe | mixture | 1 | NHCOO-i-prop | |
| | | | | cis/trans | | | |
| 2.12 | CF₃ | CF₃ | F | mixture | 0 | NHCOO-i-prop | |
| | | | | cis/trans | | | |
| 2.13 | CF₃ | CF₃ | F | mixture | 0 | NHCOO-i-prop | |
| | | | | cis/trans | | | |
| 2.14 | CF₃ | CF₃ | CH₃ | cis | 0 | 2-ethyl-2H-tetrazole-5-yl | resin |
| 2.15 | CF₃ | CF₃ | CH₃ | cis | 1 | 2-ethyl-2H-tetrazole-5-yl | 150-155 |
| 2.16 | CF₃ | CF₃ | CH₃ | cis | 0 | NHCOO-i-prop | 66-67 |
| 2.17 | CF₃ | CF₃ | CH₃ | cis | 1 | NHCOO-i-prop | 188-190 |
| 2.18 | OCF₃ | OCF₃ | CH₃ | cis | 0 | 2-ethyl-2H-tetrazole-5-yl | resin |
| 2.19 | OCF₃ | OCF₃ | CH₃ | cis | 1 | 2-ethyl-2H-tetrazole-5-yl | 138-142 |
| 2.20 | OCF₃ | OCF₃ | CH₃ | cis | 0 | NHCOO-i-prop | 67-70 |
| 2.21 | OCF₃ | OCF₃ | CH₃ | cis | 1 | NHCOO-i-prop | 188-191 |
| 2.22 | CF₃ | CF₃ | CH₃ | cis | 0 | NHCOO-Et | resin |
| 2.23 | CF₃ | CF₃ | CH₃ | cis | 1 | NHCOO-Et | 193-196 |
| 2.24 | F | F | CH₃ | cis | 0 | NHCOO-i-prop | resin |
| 2.25 | F | F | CH₃ | cis | 0 | 2-ethyl-2H-tetrazole-5-yl | resin |
| 2.26 | Cl | Cl | CH₃ | cis | 0 | 2-ethyl-2H-tetrazole-5-yl | resin |
| 2.27 | F | F | CH₃ | cis | 1 | 2-ethyl-2H-tetrazole-5-yl | 150-154 |
| 2.28 | F | F | CH₃ | cis | 1 | NHCOO-i-prop | 167-172 |
| 2.29 | Cl | Cl | CH₃ | cis | 1 | 2-ethyl-2H-tetrazole-5-yl | 154-158 |
| 2.30 | Cl | Cl | CH₃ | cis | 0 | NHCOO-i-prop | Resin |
| 2.31 | Cl | Cl | CH₃ | cis | 1 | NHCOO-i-prop | 174-182 |
| 2.32 | CF₃ | CF₃ | CH₃ | cis | 0 | 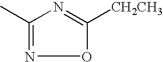 | Resin |
| 2.33 | CF₃ | CF₃ | CH₃ | cis | 1 | 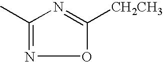 | 148-151 |
| 2.34 | CF₃ | CF₃ | CH₃ | cis | 0 | H | Resin |
| 2.35 | CF₃ | CF₃ | CH₃ | cis | 0 | NO₂ | Resin |
| 2.36 | CF₃ | CF₃ | CH₃ | cis | 0 | NH₂ | Resin |
| 2.37 | CF₃ | CF₃ | CH₃ | cis | 0 | —NHCO-2-Cl-phenyl | 82-87 |
| 2.38 | CF₃ | CF₃ | CH₃ | cis | 0 | 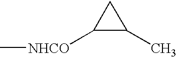 | Resin |
| 2.39 | CF₃ | CF₃ | CH₃ | cis | 1 | 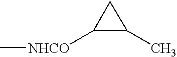 | 185-187 |
| 2.40 | CF₃ | CF₃ | CH₃ | cis | 1 | —NHCO-2-Cl-phenyl | 170-173 |
| 2.41 | CF₃ | CF₃ | CH₃ | cis | 0 | —NHCO—NH—Et | 140-142 |
| 2.42 | CF₃ | CF₃ | CH₃ | cis | 1 | —NHCO—NH—Et | 187-189 |
| 2.43 | CF₃ | CF₃ | Et | cis | 0 | 2-ethyl-2H-tetrazole-5-yl | Resin |
| 2.44 | CF₃ | CF₃ | Et | cis | 1 | 2-ethyl-2H-tetrazole-t-yl | 145-147 |
| 2.45 | CF₃ | CF₃ | Et | cis | 0 | NHCOO-i-prop | Resin |
| 2.46 | CF₃ | CF₃ | Et | cis | 1 | NHCOO-i-prop | 167-169 |
| 2.47 | CF₃ | CF₃ | CH₃ | cis | 0 | NHCOO-Me | resin |
| 2.48 | CF₃ | CF₃ | CH₃ | cis | 1 | NHCOO-Me | 155-157 |
| 2.49 | OCF₃ | OCF₃ | CH₃ | cis | 0 | NHCOO-Me | |
| 2.50 | OCF₃ | OCF₃ | CH₃ | cis | 1 | NHCOO-Me | |
| 2.51 | CF₃ | OCF₃ | CH₃ | cis | 0 | NHCOO-i-prop | |
| 2.52 | CF₃ | OCF₃ | CH₃ | cis | 1 | NHCOO-i-prop | |
| 2.53 | CF₃ | OCF₃ | CH₃ | cis | 0 | 2-ethyl-2H-tetrazole-5-yl | |
| 2.54 | CF₃ | OCF₃ | CH₃ | cis | 1 | 2-ethyl-2H-tetrazole-5-yl | |
| 2.55 | CF₃ | CF₃ | Prop | cis | 0 | NHCOO-i-prop | |
| 2.56 | CF₃ | CF₃ | Prop | cis | 1 | NHCOO-i-prop | |
| 2.57 | CF₃ | CF₃ | Prop | cis | 0 | 2-ethyl-2H-tetrazole-5-yl | |
| 2.58 | CF₃ | CF₃ | Prop | cis | 1 | 2-ethyl-2H-tetrazole-5-yl | |
| 2.59 | CF₃ | CF₃ | CH₃ | trans | 0 | 2-ethyl-2H-tetrazole-5-yl | Resin |
| 2.60 | CF₃ | CF₃ | CH₃ | trans | 1 | 2-ethyl-2H-tetrazole-5-yl | 177-179 |
| 2.61 | OCF₃ | OCF₃ | CH₃ | trans | 0 | NHCOO-i-prop | 79-81 |
| 2.62 | OCF₃ | OCF₃ | CH₃ | trans | 1 | NHCOO-i-prop | 148-152 |
| 2.63 | OCF₃ | OCF₃ | CH₃ | trans | 0 | 2-ethyl-2H-tetrazole-5-yl | 68-70 |
| 2.64 | OCF₃ | OCF₃ | CH₃ | trans | 1 | 2-ethyl-2H-tetrazole-5-yl | 138-142 |

### FORMULATION EXAMPLES

### %=Percent by Weight

| Example F1: Emulsifiable concentrates | a) | b) | c) |
|---|---|---|---|
| active ingredient | 25% | 40% | 50% |
| calcium dodecylbenzenesulfonate | 5% | 8% | 6% |
| castor oil polyethylene glycol ether | 5% | — | — |
| (36 mol EO) | | | |
| tributylphenol polyethylene glycol ether | — | 12% | 4% |
| (30 mol EO) | | | |
| cyclohexanone | — | 15% | 20% |
| xylene mixture | 65% | 25% | 20% |

Mixing finely ground active ingredient and additives gives an emulsifiable concentrate which yields emulsions of the desired concentration on dilution with water.

| Example F2: Solutions | a) | b) | c) | d) |
|---|---|---|---|---|
| active ingredient | 80% | 10% | 5% | 95% |
| ethylene glycol monomethyl ether | 20% | — | — | — |
| polyethylene glycol (MW 400) | — | 70% | — | — |
| N-methylpyrrolid-2-one | — | 20% | — | — |
| epoxidised coconut oil | — | — | 1% | 5% |
| benzine (boiling range: 160-190°) | — | — | 94% | — |

Mixing finely ground active ingredient and additives gives a solution suitable for use in the form of microdrops.

| Example F3: Granules | a) | b) | c) | d) |
|---|---|---|---|---|
| active ingredient | 5% | 10% | 8% | 21% |
| kaolin | 94% | — | 79% | 54% |
| highly dispersed silicic acid | 1% | — | 13% | 7% |
| attapulgite | — | 90% | — | 18% |

The active ingredient is dissolved in dichloromethane, the solution is sprayed onto the carrier mixture and the solvent is evaporated off in vacuo.

### BIOLOGICAL EXAMPLES

### Example B1

### Action against Heliothis Virescens Caterpillars

Young soybean plants are sprayed with an aqueous emulsion spray mixture comprising 400 ppm of test compound. After the spray-coating has dried, the soybean plants are populated with 10 caterpillars of Heliothis virescens in the first stage and placed in a plastics container. Evaluation is made 6 days later. The percentage reduction in population and the percentage reduction in feeding damage (% activity) are determined by comparing the number of dead caterpillars and the feeding damage on the treated plants with that on untreated plants.

The compounds of the Tables exhibit good activity against Heliothis virescens in this test. In particular, compounds 1.9, 2.2, 2.4, 2.5 and 2.6 are more than 80% effective.

### Example B2

### Action Against Plutella Xylostella Caterpillars

Young cabbage plants are sprayed with an aqueous emulsion spray mixture comprising 400 ppm of test compound. After the spray-coating has dried, the cabbage plants are populated with 10 caterpillars of Plutella xylostella in the third stage and placed in a plastics container. Evaluation is made 3 days later. The percentage reduction in population and the percentage reduction in feeding damage (% activity) are determined by comparing the number of dead caterpillars and the feeding damage on the treated plants with that on the untreated plants.

The compounds of the Tables exhibit good activity against Plutella xylostella in this test. In particular, compounds 1.9, 2.2, 2.4, 2.5 and 2.6 are more than 80% effective.

### Example B3

### Action Against Diabrotica Balteata Caterpillars

Maize seedlings are sprayed with an aqueous emulsion spray mixture comprising 400 ppm of the test compound. After the spray-coating has dried, the maize seedlings are populated with 10 Diabrotica balteata larvae in the second stage and placed in a plastics container. Evaluation is made 6 days later. The percentage reduction in population (% activity) is determined by comparing the number of dead larvae on the treated plants with that on untreated plants.

The compounds of the Tables exhibit good activity against Diabrotica balteata in this test. In particular, compounds 1.9, 2.2, 2.4, 2.5 and 2.6 are more than 80% effective.

### Example B4

### Action Against Spodoptera Littoralis

Young soybean plants are sprayed with an aqueous emulsion spray mixture comprising 400 ppm of test compound. After the spray-coating has dried, the soybean plants are populated with 10 caterpillars of Spodoptera littoralis in the third stage and placed in a plastics container. Evaluation is made 3 days later. The percentage reduction in population and the percentage reduction in feeding damage (% activity) are determined by comparing the number of dead caterpillars and the feeding damage on the treated plants with that on untreated plants.

The compounds of the Tables exhibit good activity against Spodoptera littoralis in this test. In particular, compounds 1.9, 2.2, 2.4, 2.5 and 2.6 are more than 80% effective.

1. A compound of formula 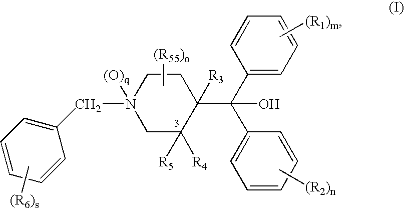
wherein
R₁ and R₂ are each independently of the other hydrogen, halogen, C₁-C₆alkyl, C₃-C₆-cycloalkyl, halo-C₁-C₆alkyl, halo-C₃-C₆cycloalkyl, C₂-C₄alkenyl, C₂-C₄alkynyl, halo-C₂-C₄alkenyl, halo-C₂-C₄alkynyl, C₁-C₆alkoxy, halo-C₁-C₆alkoxy, C₂-C₆alkenyloxy, C₂-C₆alkynyloxy, halo-C₂-C₆alkenyloxy, halo-C₂-C₆alkynyloxy, —SF₅, —C(═O)N(R₇)₂, —O—C(═O)N(R₇)₂, —CN, —NO₂, —S(═O)₂N(R₇)₂, —S(═O)P—C₁-C₆alkyl, —S(═O)ₚ-halo-C₁-C₆alkyl, —O—S(═O)ₚ—C₁-C₆alkyl, —O—S(═O)ₚ-halo-C₁-C₆alkyl, phenyl, benzyl, phenoxy or benzyloxy, wherein each of the phenyl, benzyl, phenoxy or benzyloxy radicals is unsubstituted or is substituted in the aromatic ring by from one to five substituents selected independently of one another from the group consisting of halogen, cyano, NO₂, C₁-C₆alkyl, halo-C₁-C₆alkyl, C₁-C₆alkoxy and halo-C₁-C₆alkoxy;
R₃ and R₄ are hydrogen or together form a bond;
R₅ is C₁-C₆alkyl, halo-C₁-C₆alkyl, C₃-C₆cycloalkyl, C₂-C₄alkenyl, C₂-C₄alkynyl, C₁-C₆-alkoxy, C₁-C₆alkoxyalkyl, halo-C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, halogen or hydroxy;
R₅₅ is hydrogen, C₁-C₆alkyl, halo-C₁-C₆alkyl, C₃-C₆cycloalkyl, C₂-C₄alkenyl, C₂-C₄alkynyl, C₁-C₆alkoxy, C₁-C₆alkoxyalkyl, halo-C₁-C₆alkoxy, C₂-C₆alkenyloxy or C₂-C₆alkynyloxy;
R₆ is hydrogen, halogen, CN, NO₂, C₁-C₆alkyl, halo-C₁-C₆alkyl, C₃-C₆cycloalkyl, halo-C₃-C₆cycloalkyl, C₃-C₆cycloalkoxy, C₁-C₆alkoxy, halo-C₁-C₆alkoxy, C₂-C₄alkenyl, C₂-C₄-alkynyl, halo-C₂-C₄alkenyl, halo-C₂-C₄alkynyl, C₂-C₆alkenyloxy, C₂-C₆alkynyloxy, halo-C₂-C₆alkenyloxy, halo-C₂-C₆alkynyloxy, —C(═O)-C₁-C₆alkyl, —C(═O)-halo-C₁-C₆alkyl, —C(═O)—OC₁-C₆alkyl, —C(═O)—O-halo-C₁-C₆alkyl, —N(R₇)₂, —C(═O)N(R₇)₂, —O—C(═O)N(R₇)₂, —S(═O)₂N(R₇)₂, —S(═O)ₚ—C₁-C₆alkyl, —S(═O)ₚ-halo-C₁-C₆alkyl, —O—S(═O)ₚ—C₁-C₆alkyl, —O—S(═O)ₚ-halo-C₁-C₆alkyl, —NR₁₂—C(═Y)—Z—R₁₃, —C(R₉)═N—W—R₁₀, benzyl, phenoxy, benzyloxy; or phenyl, benzyl, phenoxy, benzyloxy, heterocyclyl or heterocyclyloxy each of which is substituted by from one to five substituents selected independently of one another from the group consisting of halogen, cyano, NO₂, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈cycloalkoxy, C₃-C₈cycloalkoxy-C₁-C₆alkyl, C₃-C₈cycloalkyl-C₁-C₆alkoxy, halo-C₁-C₆alkoxy, C₂-C₄alkenyl, C₂-C₄alkynyl, halo-C₂-C₄alkenyl, halo-C₂-C₄alkynyl, C₂-C₆alkenyloxy, C₂-C₆alkynyloxy, halo-C₂-C₆alkenyloxy, halo-C₂-C₆alkynyloxy, —N(R₈)₂, phenyl, benzyl, phenoxy, benzyloxy, heterocyclyl and heterocyclyloxy;
the two R₇ radicals are each independently of the other hydrogen, C₁-C₁₋₂alkyl, halo-C₁-C₁₂alkyl, C₂-C₁₂alkenyl, halo-C₂-C₁₂alkenyl, C₂-C₁₂alkynyl, halo-C₂-C₁₂alkynyl, —C(═O)—R₁₀, —C(═S)—R₁₀, —C(═O)—O—R₁₀, —C(═S)—O—R₁₀, —C(═O)—NR₁OR₁₁, —C(═S)—NR₁₀R₁₁, —S(═O)ₚ—R₁₀, C₃-C₈cycloalkyl, aryl, aryl-C₁-C₆alkyl, heterocyclyl, heterocyclyl-C₁-C₆alkyl; or C₃-C₈cycloalkyl, aryl, aryl-C₁-C₆alkyl, heterocyclyl or heterocyclyl-C₁-C₆alkyl which, depending upon the possibilities of substitution, are each substituted in the ring by from one to five substituents selected independently of one another from halogen, hydroxy, cyano, nitro, C₁-C₆alkyl, halo-C₁-C₆alkyl, C₁-C₆alkoxy and halo-C₁-C₆alkoxy; or
together, with the nitrogen atom to which they are bonded, form a heterocyclic ring that is unsubstituted or substituted;
R₈ is hydrogen, C₁-C₆alkyl or benzyl;
R₉ is halogen, C₁-C₆alkyl, C₃-C₈cycloalkyl, C₃-C₈cycloalkyl-C₁-C₆alkyl, halo-C₁-C₆alkyl, C₁-C₆alkoxy, C₃-C₈cycloalkoxy, C₃-C₈cycloalkoxy-C₁-C₆alkyl, halo-C₁-C₆alkoxy, —NH(C₁-C₆alkyl) or —N(C₁-C₆alkyl)₂;
R₁₀ and R₁₁ are each independently of the other hydrogen, C₁-C₆alkyl, C₃-C₈cycloalkyl, C₃-C₈cycloalkyl-C₁-C₆alkyl, halo-C₁-C₆alkyl, C₂-C₄alkenyl, C₂-C₄alkynyl, halo-C₂-C₄alkenyl, halo-C₂-C₄alkynyl or —C(═O)-C₁-C₆alkyl;
R₁₂ is hydrogen, C₁-C₆alkyl, C₁-C₆alkoxy-C₁-C₆alkyl, C₃-C₈cycloalkyl, halo-C₁-C₆alkyl, C₂-C₆alkenyl or C₂-C₆alkynyl;
R₁₃ is hydrogen, C₁-C₆alkyl, halo-C₁-C₆alkyl, C₁-C₆alkoxy-C₁-C₆alkyl, C₃-C₈cycloalkyl, halo-C₁-C₆alkyl, halo-C₃-C₈cycloalkyl, C₂-C₆alkenyl, C₂-C₆alkynyl, halo-C₂-C₆alkenyl, halo-C₂-C₆alkynyl, aryl, aryl-C₁-C₆alkyl or heterocyclyl, or aryl, aryl-C₁-C₆alkyl or heterocyclyl each of which is substituted by from one to three substituents selected from the group consisting of halogen, cyano, NO₂, C₁-C₆alkyl, C₃-C₈cycloalkyl, halo-C₁-C₆alkyl, C₁-C₆alkoxy, halo-C₁-C₆alkoxy, C₂-C₄alkenyl, C₂-C₄alkynyl, halo-C₂-C₄alkenyl, halo-C₂-C₄alkynyl, C₂-C₆alkenyloxy and C₂-C₆alkynyloxy;
m is 1, 2, 3, 4 or 5;
n is 1, 2, 3, 4 or 5;
o is 1, 2 or 3;
p is 0, 1 or 2;
q is 0 or 1;
s is 1, 2, 3, 4 or 5;
Y is O or S;
Z is a bond, O, S or NR₁₄;
R₁₄ is hydrogen, C₁-C₆alkyl, C₁-C₆alkoxy-C₁-C₆alkyl, C₃-C₈cycloalkyl, halo-C₁-C₆alkyl, C₂-C₆alkenyl or C₂-C₆alkynyl;
W is O or NH or N-C₁-C₆alkyl;
or, where applicable, an E/Z isomer, a mixture of E/Z isomers and/or a tautomer, in each case in free form or in salt form.

2. A compound according to claim 1 of formula (I) wherein R₃ and R₅ are cis to each other.

3. A compound according to claim 1 of formula (I) wherein R₃ and R₄ are hydrogen.

4. A compound according to claim 3 of formula (1) wherein R₅ is C₁-C₆alkyl or halo-C₁-C₆alkyl.

5. A pesticidal composition comprising as active ingredient at least one compound of formula (I) as described in claim 1, in free form or in agrochemically acceptable form, and at least one adjuvant.

6. A process for the preparation of a composition as described in claim 5, which comprises intimately mixing the active ingredient with the adjuvant(s).

7. A method of controlling pests, which comprises applying as active ingredient a compound according to claim 1 of formula (I), in free form or, where applicable, in agrochemically acceptable salt form, to the pests or to the locus thereof.

8. Canceled.
